(19) United States
(12) Patent Application Publication
(10) Pub. No.: US 2016/0015325 A1
(43) Pub. Date: Jan. 21, 2016

(54) Personal medical product design method based on audio port

(71) Applicant: Zhufa LIN, Nanjing, Jiangsu (CN)

(72) Inventor: Zhufa Lin, Nanjing, Jiangsu (CN)

(21) Appl. No.: 14/775,601

(22) PCT Filed: Jan. 20, 2014
(86) PCT No.: PCT/CN2014/070863

§ 371 Date: Sep. 11, 2015

(30) Foreign Application Priority Data
Mar. 12, 2013 (CN) 201310077759.5

(51) Int. Cl. A61B 5/00; A61B 5/1455

(52) U.S. Cl. CPC A61B 5/6898; A61B 5/725; A61B 5/743; A61B 5/0022; A61B 5/7257; A61B 5/726; A61B 5/14552; A61B 2560/0214; A61B 2562/227; A61B 2560/0475

(57) ABSTRACT

A personal medical product design method based on an audio port includes steps of: using a universal mobile terminal and a vital sign collecting apparatus, using the mobile terminal for supplying a power for a collecting apparatus and driving the collecting apparatus to collect a vital sign signal, and then receiving the vital sign signal and performing subsequent processing; additionally mounting a physical hardware with a standard audio port on the collecting apparatus, and respectively connecting a data signal output terminal, a control signal input terminal and a power input terminal of the collecting apparatus to wire connecting terminals of the standard audio port; achieving a power supply from the mobile terminal to the collecting apparatus because a power of an audio signal outputted by the mobile terminal meets operating power requirements of the collecting apparatus; and physically connecting the collecting apparatus to the mobile terminal by the standard audio port, wherein a left sound channel signal transmission line, a right sound channel signal transmission line and a microphone signal transmission line of the audio port are respectively adapted for undertaking a power supply transmission and a signal transmission.

### CROSS REFERENCE OF RELATED APPLICATION

This is a U.S. National Stage under 35 U.S.C 371 of the International Application PCT/CN2014/070863, filed Jan. 20, 2014, which claims priority under 35 U.S.C. 119(a-d) to CN 201310077759.5, filed Mar. 12, 2013.

### BACKGROUND OF THE PRESENT INVENTION

1. Field of Invention

The present invention relates to the personal medical application field, and more particularly to a personal medical product design method based on an audio port.

2. Description of Related Arts

Currently, with the social development and improvement of living standards, people become more and more concerned about their own health. Then a lot of personal medical products based on the vital sign collecting apparatus appear, such as portable oximeter, blood glucose meter, fetal heart meter and electrocardiogram equipment. The currently personal medical product comprises a data collecting module, a calculating module, a display module and a power module. It has some disadvantages of 1) high cost; 2) large size; 3) lack of data storing and analyzing function; and 4) lack of remote data transmission function. Simultaneously, the mobile terminal, represented by smart mobile phone, is increasingly popular, it has not only strong calculating and displaying function, but power supplying, data storing analysis and remote transmission function.

### SUMMARY OF THE PRESENT INVENTION

To resolve above shortcomings of the currently personal medical product, the present invention provides a personal medical product design method based on an audio port, for reducing product cost and product size, and providing data storing analysis and remote transmission function.

Accordingly, in order to accomplish the above object, the present invention provides personal medical product design method based on an audio port, whose design idea is: using a universal mobile terminal (such as smart mobile phone, PDA and portable computer) and a vital sign collecting apparatus (which is able to be changed by the existing vital sign collecting apparatus), using the mobile terminal for supplying a power for a collecting apparatus and driving the collecting apparatus to collect a vital sign signal, and then receiving the vital sign signal and calculating, displaying, data storing, analyzing and remote transmitting;

additionally mounting a physical hardware with a standard audio port on the collecting apparatus, and respectively connecting a data signal output terminal, a control signal input terminal and a power input terminal of the collecting apparatus to wire connecting terminals of the standard audio port; and

physically connecting the collecting apparatus to the mobile terminal by the standard audio port, wherein a left sound channel signal transmission line, a right sound channel signal transmission line and a microphone signal transmission line of the audio port are respectively adapted for undertaking a power supply transmission and a signal transmission.

Based on the above design idea, the specifically technical solution of the method is as follows: (this paragraph is able to be different from the claims)

The mobile terminal has an audio hardware and an application software, wherein the application software comprises a power driving module, a sensing drive module, a sampling filter module, a calculating module, a data storing module, a data analyzing module, a display module and a remote transmission module;

step 1 is providing a power supply:

the power driving module of the mobile terminal outputs a sine wave with a certain frequency by a left or right sound channel of a sound card of the mobile terminal to a collecting apparatus, the sine wave has an audio file corresponding to the sine frequency, the power module of the collecting apparatus processes the sine wave and then provides a stable power output;

step 2 is collecting work control of the collecting apparatus:

the sensing drive module of the mobile terminal generates a square wave, the square wave has an audio file corresponding to the square wave, the square wave is transmitted to a control signal input terminal of the collecting apparatus by a sound channel different from a sound channel adopted by an output of the power driving module;

step 3 is collecting a vital sign signal:

the control module of the collecting apparatus controls the collecting work via a rising edge or a falling edge of the square wave;

step 4 is processing the vital sign signal:

the vital sign signal collected by the collecting apparatus is sent to a microphone signal input terminal of the mobile terminal by a microphone signal transmission line;

step 5 is sampling and filtering the vital sign signal:

the data signal from the microphone signal transmission line is processed by the sampling filter module of the mobile terminal for obtaining a desired signal;

step 6 is calculating:

the data signal is calculated by the calculating module of the mobile terminal for obtaining a value which is capable of reflecting the vital sign;

step 7 is storing data:

the value obtained by calculating is stored by the data storing module of the mobile terminal;

step 8 is analyzing data:

historical data in the data storing module of the mobile terminal are firstly made statistics and then the statistic data are analyzed, and then an analyzing result is sent to a storing space of the mobile terminal by the storing module;

Step 9 is displaying data:

the data are taken out of the spacing space of the mobile terminal by the data display module of the mobile terminal, and then currently collected real-time data are displayed on a screen of the mobile terminal, and then a result after data analyzing is displayed on the screen by a report form or graphics mode;

step 10 is remote data transmission:

the remote communication module of the mobile terminal is connected to Internet via GPRS module, 3G module or WIFI module of the mobile terminal for transmitting the collected data to a remote server in real time or bulk.

As an further improvement, in the step 1, the processing procedure of the power module of the collecting apparatus is as follows: the sine wave is firstly step-up by a step-up transformer, and then FET rectified, and finally voltage stabilized by a blocking diode and a filter capacitor for achieving the stable power output, so as to provide the power for the collecting apparatus;

wherein it is the key for the power module that the rectifier circuit has a dead zone pressure drop in a low-pressure system, if a low-pressure diode is used during a rectification process, it is found in actual measurement that most of the power are consumed, and only little of the power are transmitted to a load, if the diode is replaced to by an FET, a synchronous rectifying is generally used to reduce the loss.

In the step 5, the signal from the microphone signal transmission line is sampled by the sampling filter module of the mobile terminal, which specifically comprises steps of:

firstly sampling the vital sign signal from a microphone channel at a certain sampling rate, and then making a signal processing, wherein the signal processing adopts an IIR filter and/or FIR filter to digitally filter; extracting a DC (direct current) component and an AC (alternating current) component of a sampled result, wherein the DC component is tracked by the IIR filter, and then the DC component is removed from an analog signal of the inputted vital sign signal for obtaining the AC component; and making a band-pass filtering by a band-pass FIR filter, wherein Fourier transform or wavelet transform complex algorithm is used according to actual requirements.

In the step 7, data are stored by the data storing module with a data compression algorithm that if measurement values in a certain period are same, then they are stored by a record with attributes comprising beginning time, ending time, number of measurement and measurement value.

### BRIEF DESCRIPTION OF THE DRAWINGS

FIG. 1 is a structurally schematic view of a blood oxygen vital sign measurement system based on an audio port according to a preferred embodiment of the present invention.

FIG. 2 is a structural diagram of the blood oxygen vital sign measurement system according to the preferred embodiment of the present invention.

FIG. 3 is a basic flow chart of the blood oxygen vital sign measurement system according to the preferred embodiment of the present invention.

FIG. 4 is circuit schematic diagram of a power module of the blood oxygen vital sign measurement system according to the preferred embodiment of the present invention.

FIG. 5 is a circuit schematic diagram of an LED control module of the blood oxygen vital sign measurement system according to the preferred embodiment of the present invention.

FIG. 6 is a circuit schematic diagram of a PIN signal processing module of the blood oxygen vital sign measurement system according to the preferred embodiment of the present invention.

### DETAILED DESCRIPTION OF THE PREFERRED EMBODIMENT

A personal medical product design method based on an audio port comprises steps of: using a universal mobile terminal and a vital sign collecting apparatus, using the mobile terminal for supplying a power for a collecting apparatus and driving the collecting apparatus to collect a vital sign signal, and then receiving the vital sign signal and performing subsequent processing; additionally mounting a physical hardware with a standard audio port on the collecting apparatus, and respectively connecting a data signal output terminal, a control signal input terminal and a power input terminal of the collecting apparatus to wire connecting terminals of the standard audio port; achieving a power supply from the mobile terminal to the collecting apparatus because a power of an audio signal outputted by the mobile terminal meets operating power requirements of the collecting apparatus; and physically connecting the collecting apparatus to the mobile terminal by the standard audio port, wherein a left sound channel signal transmission line, a right sound channel signal transmission line and a microphone signal transmission line of the audio port are respectively adapted for undertaking a power supply transmission and a signal transmission.

The mobile terminal performing subsequent processing after receiving the vital sign signal comprises calculating, displaying, data storing, analyzing and remote transmission processing of the received vital sign signal.

The collecting apparatus comprises a power module, a sensor control module, a sensor, and a sensor signal processing module; the mobile terminal has an audio hardware and an application software, wherein the application software comprises a power driving module, a sensing drive module, a sampling filter module, a calculating module, a data storing module, a data analyzing module, a display module and a remote transmission module.

Step 1: providing a power supply

The power driving module of the mobile terminal outputs a signal wave with a certain frequency by a left or right sound channel of a sound card of the mobile terminal to the collecting apparatus. The signal wave has an audio file corresponding to the certain frequency. The power module of the collecting apparatus processes the signal wave and then provides a stable power output.

Step 2: Collecting work control of the collecting apparatus

Mode 1 (analog signal mode): The sensing drive module of the mobile terminal generates a control signal, wherein the control signal is a square wave, the square wave has a corresponding audio file; the square wave is transmitted to the control signal input terminal of the collecting apparatus by a sound channel different from a sound channel adopted by an output of the power driving module; or

Mode 2 (digital signal mode): The sensing drive module of the mobile terminal adopts a serial communication mode to transmit a control command, which is equivalent to the control signal in the mode 1. In the digital circuit, a term “command” is generally adopted (the situation of complex control means that information communication is needed).

Step 3: Collecting the vital sign signal

Corresponding to the mode 1 of the step 2, the sensor is controlled by the sensor control module of the collecting apparatus via a rising edge or a falling edge of the square wave.

Corresponding to the mode 2 of the step 2, the control command is received by the serial communication mode, and the sensor is controlled by a microprocessor.

Step 4: Processing the vital sign signal

The vital sign signal collected by the sensor is processed by the sensor signal processing module of the collecting apparatus, and then sent to a microphone signal input terminal of the mobile terminal by the microphone signal transmission line.

Step 5: Sampling and filtering the vital sign signal

The data signal from the microphone signal transmission line is processed by the sampling filter module of the mobile terminal for obtaining a desired signal.

Step 6: Calculating

The data signal is calculated by the calculating module of the mobile terminal for obtaining a value which is capable of reflecting the vital sign.

Step 7: Storing data

The value obtained by calculating is stored by the data storing module of the mobile terminal

Step 8: Analyzing data

Historical data in the data storing module are firstly made statistics and then analyzed by the data analyzing module of the mobile terminal, and then an analyzing result is sent to a storing space of the mobile terminal by the storing module.

Step 9: Displaying data

The data are taken out of the spacing space of the mobile terminal by the display module of the mobile terminal, and then currently collected real-time data are displayed on a screen of the mobile terminal, and then a result after data analyzing is displayed on the screen by a report form or graphics mode.

Step 10: Remote data transmission

The remote communication module of the mobile terminal is connected to Internet via GPRS module, 3G module or WIFI module of the mobile terminal for transmitting the collected data to a remote server in real time or bulk.

In the step 1, the signal wave is a sine wave or a square wave.

In the step 4, corresponding to two modes in step 2 and step 3, there are three signal processing modes:

a) corresponding to the mode 1: after analog signal processing, an analog signal is directly transmitted to the mobile terminal;

b) corresponding to the mode 2: a digital signal is obtained by the microprocessor, and then is transmitted to the mobile terminal;

c) corresponding to the mode 2: a digital signal is obtained by the microprocessor, and then a calculating result is transmitted to the mobile terminal after calculating the digital signal. At this time, filtering and data calculating in the microprocessor of the mobile terminal are moved to the collecting apparatus.

In the step 1, the operating process of the power module of the collecting apparatus is as follows. Firstly the sine wave or the square wave is step-up by a step-up transformer, and then FET rectified, and finally voltage stabilized by a blocking diode and a filter capacitor for achieving the stable power output, so as to provide the power for the collecting apparatus. The power module of the collecting apparatus comprises an Farah capacitor. The Farah capacitor, the blocking diode and the filter capacitor form a Π-shaped parallel circuit.

The Farah capacitor is capable of better meeting power requirements of the collecting apparatus, and being charged during the working gap of high-power components. While the high-power components work, the Farah capacitor and the Π-shaped circuit make the power output together for allowing the collecting apparatus to be in a good power supply state.

In the step 5, the signal from the microphone signal transmission line is sampled by the sampling filter module of the mobile terminal, which specifically comprises steps of:

firstly sampling the vital sign signal from a microphone channel at a certain sampling rate, and then making a signal processing, wherein the signal processing adopts an IIR filter and/or FIR filter to digitally filter;

extracting a DC (direct current) component and an AC (alternating current) component of a sampled result, wherein the DC component is tracked by the IIR filter, and then the DC component is removed from an analog signal of the inputted vital sign signal for obtaining the AC component; and

making a band-pass filtering by a band-pass FIR filter,

wherein the mode 2 of the step 2 is processed by Fourier transform or wavelet transform complex algorithm.

In the step 7, data are stored by the data storing module by a data compression algorithm that if measurement values in a certain period are same, then they are stored by a record with attributes comprising beginning time, ending time, number of measurement and measurement value.

The technical solution of the present invention is further described in detail combined with drawings and embodiments.

The method disclosed by the present invention is based on the vital sign collecting apparatus (hereinafter referred to as collecting apparatus). The collecting apparatus is collected to a mobile terminal by an audio port for providing the power supply and transmitting data. While transmitting data, encoding is an audio signal. The user starts a personal medical application software on the mobile terminal, the personal medical application software makes a communication handshake with the collecting apparatus by the audio port and allows the user to measure the vital sign after the collecting apparatus gaining access. While the user measures the vital sign, the application software controls the collecting apparatus by the audio port to collect the signal and receive the collected signal. The collected signal is sampled, filtered and calculated for obtaining a vital sign datum. The vital sign datum is capable of being displayed on the screen of the mobile terminal in real time and being stored the permanent storing space of the mobile terminal, thus establishing a personal medical data base and analyzing the datum based on various application requirements. Simultaneously, the datum is transmitted to other places by the remote communication module of the mobile terminal for meeting requirements such as remote real-time monitoring, remote diagnosis, and remote health analysis.

Take blood oxygen vital sign measurement as an example for further explanation as below.

An oximeter based on an audio port communication, comprises a power module, a sensor control module, a sensor, a sensor signal processing module and a physical hardware with a standard audio port;

wherein a left sound channel signal transmission line, a right sound channel signal transmission line and a microphone signal transmission line of the audio port are respectively adapted for undertaking a power supply transmission, a control signal input and a collecting signal output;

a control signal input terminal of the sensor control module is connected to a control signal input line of the audio port;

an input terminal of the power module is connected to a power supply transmission line of the audio port;

an output terminal of the sensor signal processing module is connected to a collecting signal output line of the audio port;

a control signal output terminal of the sensor control module is connected to a control signal input terminal of the sensor; and

a signal output terminal of the sensor is connected to an input terminal of the sensor signal processing module.

The power module comprises a step-up transformer, an FET rectifier circuit, a blocking diode and a filter capacitor; wherein a primary side of the step-up transformer is the input terminal of the power module; a secondary side of the step-up transformer is connected to an input terminal of the FET rectifier circuit; an output terminal of the FET rectifier circuit is connected to an input terminal of a Π-shaped circuit formed by the blocking diode and the filter capacitor, and an output terminal of the Π-shaped circuit is the output terminal of the power module.

The sensor control module is a microprocessor or an analog circuit.

The sensor comprises a PIN diode, a red LED and an infrared LED; wherein the PIN diode receives light from the red LED and the infrared LED, one end of the PIN diode is connected to the power supply, namely, the output terminal of the sensor.

(1) When the sensor control module is the microprocessor, a control signal output terminal of the microprocessor is connected to the red LED and the infrared LED by a driving circuit; simultaneously, the microprocessor acts as the sensor signal processing module, and the output terminal of the sensor is connected to a signal input terminal of the microprocessor; or

(2) When the sensor control module is the analog circuit, the analog circuit comprises:

a) 1-bit binary counter formed by a D flip-flop and an inverter; wherein a clock signal input terminal of the D flip-flop is connected to the control signal input line of the audio port, an output terminal of the D flip-flop is connected to two input terminals of the inverter, an anode of the red LED and an anode of the infrared LED are respectively connected to an input terminal and an output terminal of the inverter, and a D terminal of the D flip-flop is connected to the output terminal of the inverter; and

b) a voltage controlled constant current circuit formed by an OA (operational amplifier) and a triode; wherein a high-level input terminal of the OA is connected to the control signal input line of the audio port, an output terminal of the OA is connected to a base of the triode, a collector of the triode is connected to a cathode of the red LED and the infrared LED, and a low-level input terminal of the OA is connected to an emitter of the triode.

The other end of the PIN diode is connected to an input terminal of an amplifying circuit, an output terminal of the amplifying circuit is connected to the collecting signal output line of the audio port, and the amplifying circuit acts as the sensor signal processing module.

The microprocessor further comprises a blood oxygen output terminal. Because an operational function of the microprocessor is capable of meeting an operation from a blood oxygen signal to a blood oxygen value, the operation is able to be finished in the oximeter.

The power module further comprises an Farah capacitor. The Farah capacitor is connected to the Π-shaped parallel circuit. The Farah capacitor is capable of better meeting power requirements of the collecting apparatus, and being charged during the working gap of high-power components such as LEDs. While the LEDs work, the Farah capacitor and the Π-shaped circuit make the power output together for allowing the collecting apparatus to be in a good power supply state.

Specific description is as follows.

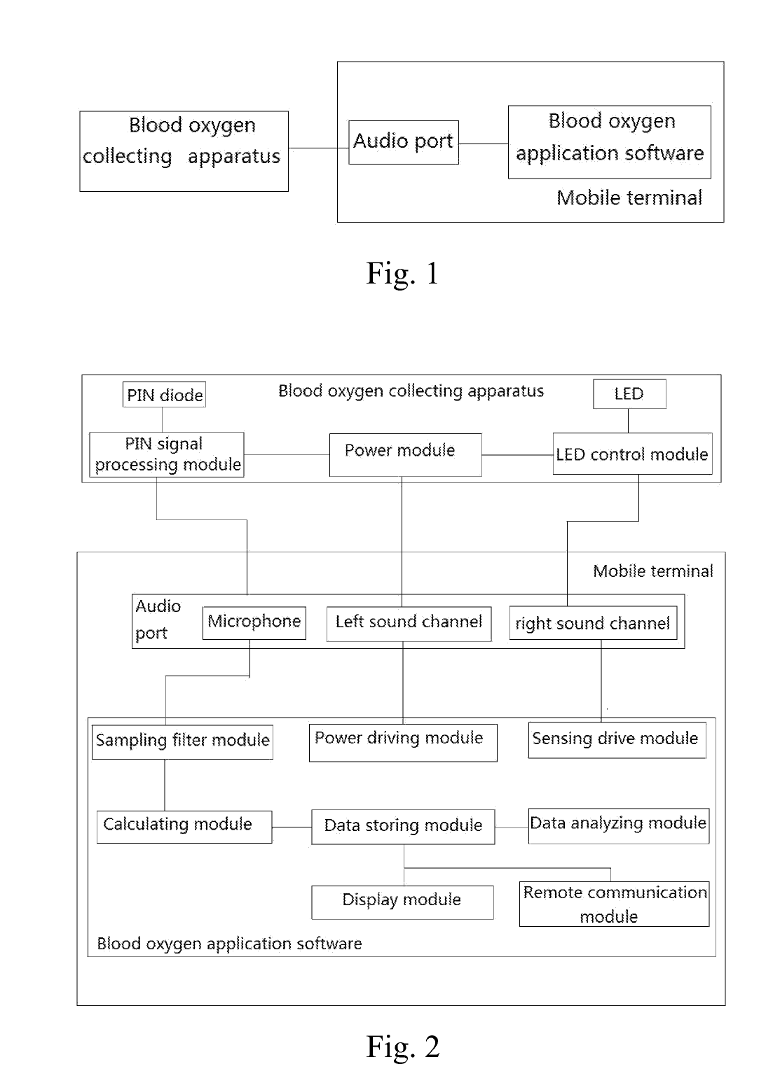

FIG. 1 is a structurally schematic view of a blood oxygen vital sign measurement system based on an audio port provided by the present invention. As shown in FIG. 1, the system comprises a blood oxygen vital sign collecting apparatus and a mobile terminal, wherein the blood oxygen vital sign collecting apparatus (hereinafter referred to as blood oxygen collecting apparatus) is connected to the mobile terminal by the audio port of the mobile terminal, a blood oxygen measurement application software (hereinafter referred to as blood oxygen application software) is installed on the mobile terminal

FIG. 2 is a structural diagram of the blood oxygen vital sign measurement system. As shown in FIG. 2, the blood oxygen collecting apparatus comprises a power module, an LED control module, a PIN signal processing module, an LED and a PIN diode; wherein the power module is connected to a left sound channel of the audio port of the mobile terminal for transforming a sine wave electrical signal, which is outputted by the audio port of the mobile terminal, into a stable voltage output, so as to provide a power output for other modules; the LED control module is connected to a right sound channel of the mobile terminal for controlling a switching and a current value of two LEDs via a square wave signal outputted by the mobile terminal, so as to control a switching and a light intensity of the red and infrared light; the PIN signal processing module is adapted for outputting an electrical signal generated by the PIN diode after transforming and amplifying to a microphone channel of the audio port of the mobile terminal.

The blood oxygen application software comprises a power driving module, a sensing drive module, a sampling filter module, a calculating module, a data storing module, a data analyzing module, and a remote transmission module; wherein the power driving module is adapted for generating a sine wave audio signal with stable frequency, and outputting to the power module of the collecting apparatus via the left sound channel of the audio port; the sensing drive module is adapted for generating a square wave signal and transmitting to the LED control module of the collecting apparatus via the right sound channel of the audio port, so as to drive the LED to generate red and infrared light; the sampling filter module is adapted for sampling an analog signal inputted via the microphone channel of the audio port, and then filtering for removing noises, and then separating a DC (direct current) component and an AC (alternating current) component of the filtered signal; the calculating module is adapted for calculating a saturation value of the blood oxygen based on the DC component, and calculating a pulse value based on the AC component; the data storing module is adapted for storing the calculated values in a permanent storing space of the mobile terminal; the data analyzing module is adapted for analyzing the collected historical data and generating a corresponding report form; and the remote communication module is adapted for transmitting the collected data to other places for meeting requirements such as remote real-time monitoring, remote diagnosis, and remote health analysis.

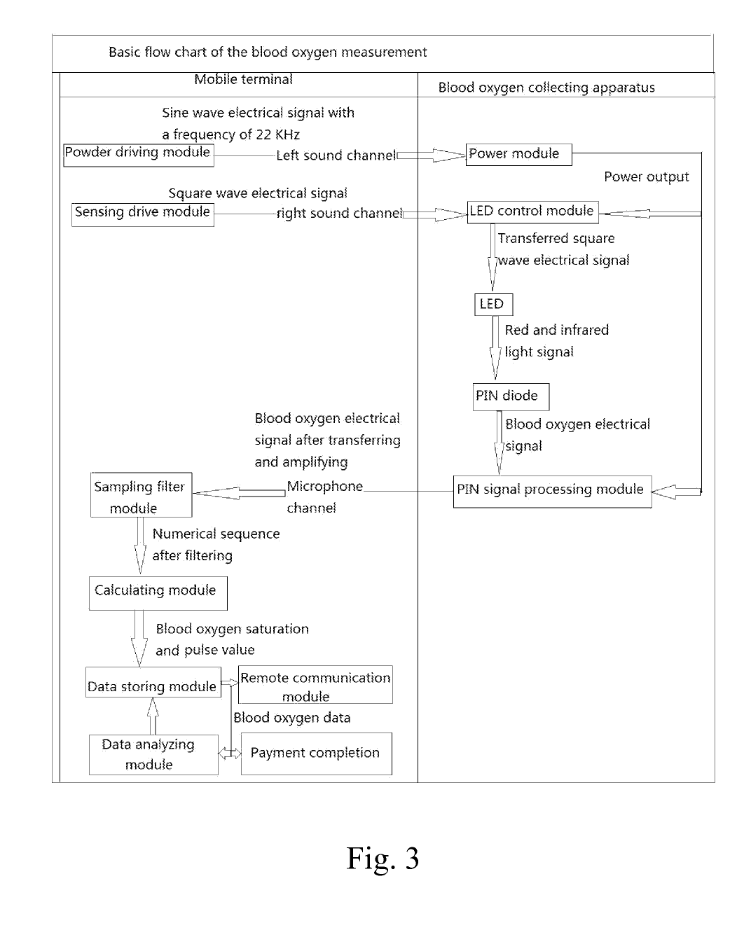

FIG. 3 is a basic flow chart of the blood oxygen vital sign measurement system. As shown in FIG. 3,

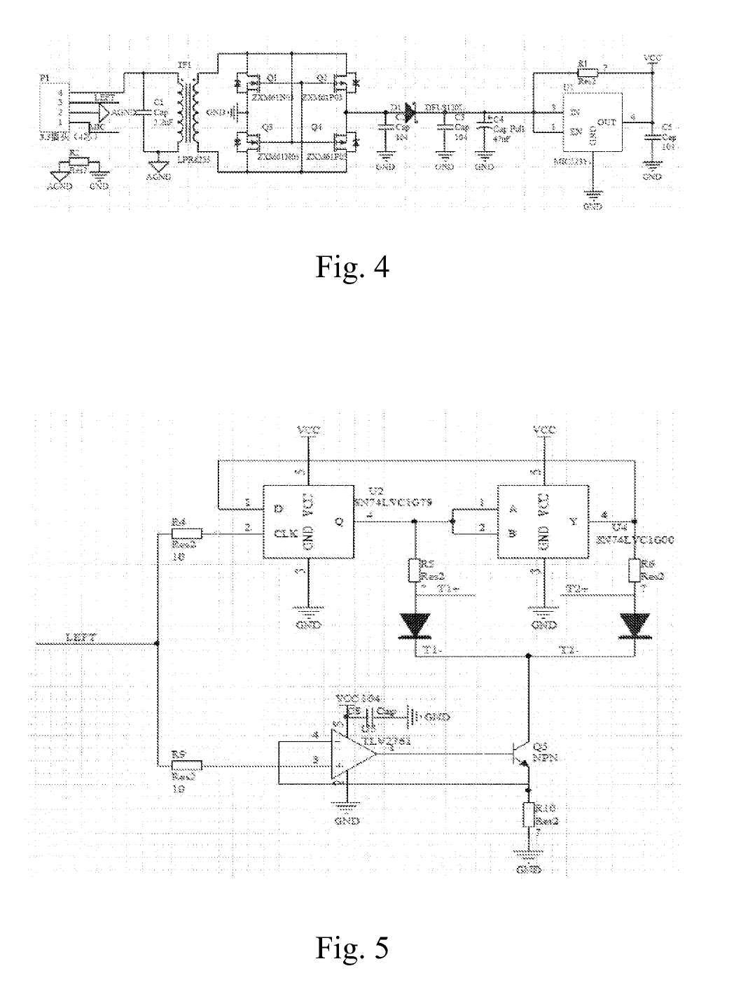

Step 1 is providing a power supply. The power driving module of the blood oxygen application software outputs a square wave with a frequency of 22 kHz by the left sound channel of the sound card of the mobile terminal to the blood oxygen collecting apparatus, which is specifically achieved by broadcasting a square wave audio file with a frequency of 22 kHz. The power module of the blood oxygen collecting apparatus serially processes the square wave and then provides a stable power output. The specific processing procedure of the power module is as follows: firstly the 22 KHz square wave is step-up by the step-up transformer, and then FET rectified, and finally voltage stabilized by the blocking diode and the filter capacitor for achieving the stable power output, so as to provide the power for other processing circuits. It is the key for the power module that the rectifier circuit has a dead zone pressure drop in a low-pressure system. To obtain a maximum power transmission, if a low-pressure diode such as DFLS120L is used during a rectification process, it is found in actual measurement that 80% of the power is consumed, and only 20% of the power is transmitted to a load. If the diode is replaced by an FET, a synchronous rectifying is generally used to reduce the loss (a circuit schematic diagram of the power module refers to FIG. 4).

Step 2 is driving and controlling LEDs. The sensing drive module of the blood oxygen application software generates the square wave, which is specifically achieved by broadcasting the square wave audio file. The square wave is transmitted to the LED control module via the right sound channel of the audio port. The LED control module utilizes a rising edge of the square wave to control the switching of the red and infrared light, and a high-level voltage of the square wave controls an exciting current value of two LEDs of the sensor. In the LED control module, a D flip-flop and an inverter forms a 1-bit binary counter for achieving the switching of the two LEDs. An OA and a triode form a voltage controlled constant current circuit for achieving a control of the exciting current value of the two LEDs. FIG. 5 is a circuit schematic diagram. (Preferably, digital signal mode is adopted to use an MCU for controlling.

Step 3 is collecting the blood oxygen vital sign. The LED module comprises two LEDs (light emitting diode). One LED emits red light (with a wavelength of 660 nm), the other emits infrared light (with a wavelength of 940 nm). The two LEDs are controlled by the LED control module to make a TDM (time division multiplexing) at 500 times per second. The PIN diode is penetrated by two different LEDs and then alternately activated to generate an electrical signal containing blood oxygen information.

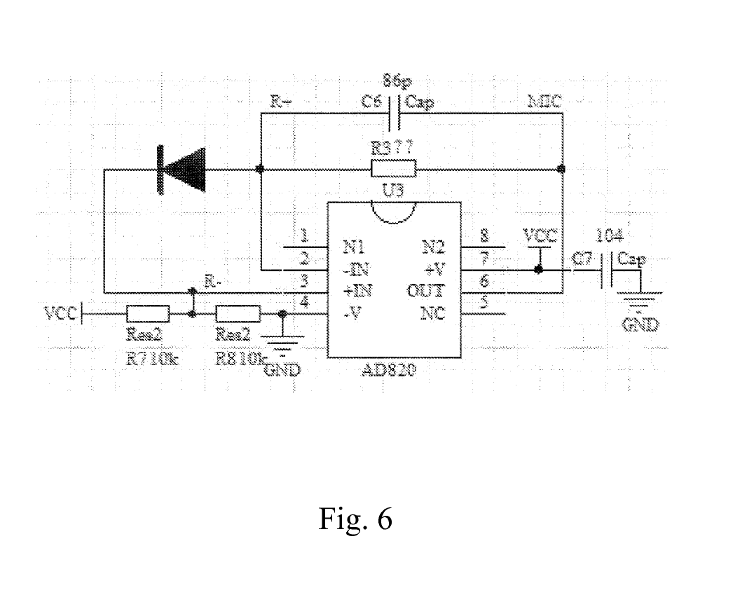

Step 4 is processing the PIN blood oxygen signal. The PIN signal processing module takes a current signal by the PIN sensor, the current signal passes through a current amplifier formed by the OA, the amplified signal as a voltage signal is sent to an input terminal of MIC of the mobile terminal; wherein the amplifier simultaneously amplifies the AC and the DC, the DC may be very large, and the AC may be very small; if an amplification at this time is over high, then the signal will enter saturation. Therefore, a proper amplification should be adopted, and a proper grey scale is given by controlling the exciting current. A circuit schematic diagram of the PIN signal processing module refers to FIG. 6.

(More preferably, a digital signal mode is adopted, wherein a digital signal is obtained by the MCU, and the digital signal is primarily processed and then transmitted to the mobile terminal).

Step 5 is sampling and filtering the blood oxygen signal. The sampling filter module of the blood oxygen application software firstly samples the blood oxygen analog signal inputted from the microphone channel at a velocity of 1000 sps; and then extracts a DC component of a sampled result, due to a required cut-off frequency is low, an IIR filter is adopted to track the DC component; and then obtains an AC component by removing the DC component from an inputted signal; and then using a corner frequency of 6 Hz, 50 Hz and higher, removes environmental noises with a frequency more than 50 Hz via a low pass FIR filter with 50 dB attenuation. At this time, the AC component signal is similar to heartbeat pulse passing through arteries.

Step 6 is calculating data. Firstly calculate a RMS value of the DC components of the red light blood oxygen signal and the infrared light blood oxygen signal, and then obtains a degree of blood oxygen saturation by taking a logarithm of the RMS value. Pulses are obtained by counting a sampling number within three beats.

Step 7: Storing data. The degree of blood oxygen saturation and the pulse value, obtained from data calculating, are stored in the built-in database of the mobile terminal. The data amount growth from blood oxygen collecting is very fast, especially under the condition of long-term continuous measurement, due to limited storing space of the mobile terminal, the key of data storing is data compression algorithm. The adopted algorithm is that: if measurement values in a certain period are same, then they are stored by a record with attributes comprising beginning time, ending time, number of measurement and measurement value, so that a plurality of measurement results are stored by one data record.

Step 8 is analyzing data. Historical data are firstly made statistics and then analyzed according to specific requirements such as sleep analysis, and then an analyzing result is sent to the storing space of the mobile terminal by the storing module.

Step 9 is displaying data. The data are taken out of the spacing space of the mobile terminal by the data display module, and then currently collected real-time data are displayed on the screen of the mobile terminal, and then a result after data analyzing is displayed on the screen by the report form or graphics mode.

Step 10 is remote data transmission. The remote communication module is connected to Internet via GPRS module, 3G module or WIFI module of the mobile terminal for transmitting the collected data to the remote server in real time or bulk, thereby achieving real-time health monitoring, remote diagnosis, remote health analysis and remote data backup.

Preferably, the steps 5 and 6 are able to be accomplished by the MCU, and then the calculating result is transmitted to the mobile terminal by serial communication via the MCU.

Finally, it should be noted that one skilled in the art will understand that the embodiment of the present invention as shown in the drawings and described above is exemplary only and not intended to be limiting. It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A personal medical product design method based on an audio port, comprising step of: using a universal mobile terminal and a vital sign collecting apparatus, using the mobile terminal for supplying a power for a collecting apparatus and driving the collecting apparatus to collect a vital sign signal, and then receiving the vital sign signal and performing subsequent processing; additionally mounting a physical hardware with a standard audio port on the collecting apparatus, and respectively connecting a data signal output terminal, a control signal input terminal and a power input terminal of the collecting apparatus to wire connecting terminals of the standard audio port; achieving a power supply from the mobile terminal to the collecting apparatus because a power of an audio signal outputted by the mobile terminal meets operating power requirements of the collecting apparatus; and physically connecting the collecting apparatus to the mobile terminal by the standard audio port, wherein a left sound channel signal transmission line, a right sound channel signal transmission line and a microphone signal transmission line of the audio port are respectively adapted for undertaking a power supply transmission and a signal transmission.

2. The personal medical product design method based on the audio port, as recited in claim 1, wherein after the mobile terminal receiving the vital sign signal, the post-processing comprises calculating, displaying, data storing, analyzing and remote transmitting of the vital sign signal.

3. The personal medical product design method based on the audio port, as recited in claim 2, wherein the collecting apparatus comprises a power module, a sensor control module, a sensor, and a sensor signal processing module;
wherein the mobile terminal has an audio hardware and an application software, wherein the application software comprises a power driving module, a sensing drive module, a sampling filter module, a calculating module, a data storing module, a data analyzing module, a display module and a remote transmission module;
wherein the method comprises steps of:
step 1) providing a power supply:
wherein the power driving module of the mobile terminal outputs a signal wave with a certain frequency by a left or right sound channel of a sound card of the mobile terminal to a collecting apparatus, the signal wave has an audio file with a corresponding frequency, the power module of the collecting apparatus processes the signal wave and then provides a stable power output;
step 2) controlling a collecting work of the collecting apparatus:
wherein mode 1: the sensing drive module of the mobile terminal generates a control signal, wherein the control signal is a square wave, the square wave has an audio file corresponding to the square wave; the square wave is transmitted to a control signal input terminal of the collecting apparatus by a sound channel different from a sound channel adopted by an output of the power driving module; or
mode 2: the sensing drive module of the mobile terminal adopts a serial communication mode to transmit a control command;
Step 3) collecting the vital sign signal:
wherein corresponding to the mode 1 of the step 2, the sensor is controlled by the sensor control module of the collecting apparatus via a rising edge or a falling edge of the square wave,
corresponding to the mode 2 of the step 2, the control command is received by the serial communication mode, and the sensor is controlled by a microprocessor;
step 4) processing the vital sign signal:
wherein the vital sign signal collected by the sensor is processed by the sensor signal processing module of the collecting apparatus, and then sent to a microphone signal input terminal of the mobile terminal by the microphone signal transmission line;
step 5) sampling and filtering the vital sign signal:
wherein the data signal from the microphone signal transmission line is processed by the sampling filter module of the mobile terminal for obtaining a desired signal;
step 6) calculating:
wherein the data signal is calculated by the calculating module of the mobile terminal for obtaining a value which is capable of reflecting the vital sign;
step 7) storing data:
wherein the value obtained by calculating is stored by the data storing module of the mobile terminal;
step 8) analyzing data:
wherein historical data in the data storing module are firstly made statistics and then analyzed by the data analyzing module of the mobile terminal, and then an analyzing result is sent to a storing space of the mobile terminal by the storing module;
step 9) displaying data:
wherein the data are taken out of the spacing space of the mobile terminal by the display module of the mobile terminal, and then currently collected real-time data are displayed on a screen of the mobile terminal, and then a result after data analyzing is displayed on the screen by a report form or graphics mode;
step 10) remote data transmission:
wherein the remote communication module of the mobile terminal is connected to Internet via GPRS module, 3G module or WIFI module of the mobile terminal for transmitting the collected data to a remote server in real time or bulk.

4. The personal medical product design method based on the audio port, as recited in claim 3, wherein in the step 1), the signal wave is a sine wave or a square wave.

5. The personal medical product design method based on the audio port, as recited in claim 3, wherein in the step 5), the signal from the microphone signal transmission line is sampled by the sampling filter module of the mobile terminal, which specifically comprises steps of:
firstly sampling the vital sign signal from a microphone channel at a certain sampling rate, and then making a signal processing, wherein the signal processing adopts an IIR filter and/or FIR filter to digitally filter;
extracting a DC (direct current) component and an AC (alternating current) component of a sampled result, wherein the DC component is tracked by the IIR filter, and then the DC component is removed from an analog signal of the inputted vital sign signal for obtaining the AC component; and
making a band-pass filtering by a band-pass FIR filter,
wherein the mode 2 of the step 2 is processed by Fourier transform or wavelet transform complex algorithm.

6. The personal medical product design method based on the audio port, as recited in claim 5, wherein in the step 4), corresponding to two modes in step 2 and step 3, there are three signal processing modes:
a) corresponding to the mode 1: after analog signal processing, an analog signal is directly transmitted to the mobile terminal;
b) corresponding to the mode 2: a digital signal is obtained by the microprocessor, and then is transmitted to the mobile terminal;
c) corresponding to the mode 2: a digital signal is obtained by the microprocessor, and then a calculating result is transmitted to the mobile terminal after calculating the digital signal; at this time, filtering and data calculating in the microprocessor of the mobile terminal are moved to the collecting apparatus.

7. The personal medical product design method based on the audio port, as recited in claim 3, wherein in the step 1), an operating process of the power module of the collecting apparatus is as follows: firstly the sine wave or the square wave is step-up by a step-up transformer, and then FET rectified, and finally voltage stabilized by a blocking diode and a filter capacitor for achieving the stable power output, so as to provide the power for the collecting apparatus.

8. The personal medical product design method based on the audio port, as recited in claim 3, wherein in the step 7), data are stored by the data storing module by a data compression algorithm that if measurement values in a certain period are same, then they are stored by a record with attributes comprising beginning time, ending time, number of measurement and measurement value.

9. The personal medical product design method based on the audio port, as recited in claim 6, wherein a power module of the collecting apparatus comprises an Farah capacitor, wherein the Farah capacitor, the blocking diode and the filter capacitor form a Π-shaped parallel circuit.
